(19) United States
(12) Patent Application Publication
(10) Pub. No.: US 2014/0066969 A1
(43) Pub. Date: Mar. 6, 2014

(54) BLOOD CLOT TREATMENT METHOD AND APPARATUS

(71) Applicant: Joe Michael Eskridge, Clyde Hill, WA (US)

(72) Inventor: Joe Michael Eskridge, Clyde Hill, WA (US)

(21) Appl. No.: 13/604,594

(22) Filed: Sep. 5, 2012

(51) Int. Cl. A61B 17/22

(52) U.S. Cl. 606/200

(57) ABSTRACT

A medical catheter that has a medial section having twin lumens, each enclosing a separately controllable wire control unit, proximal to said medial section, permitting a user to control said wires; and a fossa distal to said medial section, enclosing an expandable device that may be deployed to have a distal, clot fragment guard section and a proximal clot disrupting section and which is orientation controllable by said wires.

### BACKGROUND

Until the middle part of the twentieth century, there was very little that medical professionals could do to intervene when a patient suffered a stroke. Gradually, a range of medicinal therapies have been developed, but direct physical intervention after the event is still fairly rare. The difficulty of reaching the event site is part of the reason. The blood vessels of the brain are formed in tortuous pathways that are very difficult to navigate. The alternative effort of reaching the event site through surrounding tissue is complicated by the sensitive and critical nature of brain tissue.

Nevertheless, it is known to use a stent positioned at the end of a catheter to capture a clot or a portion of a clot and pull it through blood vessels out of the body. One problem with this method is that clot fragments can become dislodged during the procedure and travel in the direction of blood flow to some more interior portion of the brain, where a secondary stroke or strokes may occur. Also, it is difficult to impossible to steer the stent and it appears that access past the carotid artery has not been achieved, using this method.

Typically, to maneuver a clot-capture stent into a blood vessel of the brain requires a number of steps. First, an incision is made into the femoral artery and a sheath is introduced, extending approximately to the aorta. A first guide catheter is inserted through the sheath and extended up into the carotid artery. A second guide catheter is coaxially introduced through the first guide catheter and extended up into the target aneurysm. Both guide catheters are introduced using a guide wire having a steerable tip of either stainless steel or nitinol. Then, a microcatheter introducer is inserted through the guide catheter, to the clot, and the stent is placed at or into the clot. Heretofore, however, once reaching the clot there has been no effective method for positioning a device that requires precise positioning. A device that would require a definite orientation, as it is withdrawn from the body, presents particular challenges in positioning during implantation.

Another difficulty in delivering a complex device to a clot site is the lack of space to pack such a device in a lumen at the end of a microcatheter. Any such device must fold into a cylinder having an internal diameter on the order of 1 mm and a length of about 10 mm.

### SUMMARY

The following embodiments and aspects thereof are described and illustrated in conjunction with systems, tools and methods which are meant to be exemplary and illustrative, not limiting in scope. In various embodiments, one or more of the above-described problems have been reduced or eliminated, while other embodiments are directed to other improvements.

In a first, separate aspect, the present invention is a medical catheter that has a medial section having twin lumens, each enclosing a separately controllable wire control unit, proximal to the medial section, permitting a user to control the wires; and a fossa distal to the medial section, enclosing an expandable device that may be deployed to have a distal, clot fragment guard section and a proximal clot disrupting section and which is orientation controllable by the wires.

In a second separate aspect, the present invention is a medical procedure for treating a blood clot in an artery, which utilizes a medical catheter, having a distal end of the catheter, defining a fossa that encloses an expandable device that may be deployed to have a distal, clot fragment guard section and a proximal clot disrupting section and which is orientation controllable by the wires; and a control unit connected to the expandable device by a medial section, and permitting user deployment and position control of the expandable device, transmitted through the medial section. The method begins with the introduction of the distal end of the catheter into the artery and the guidance of the distal end into the clot, deploying the expandable device from the fossa so that the clot fragment guard section is upstream from the clot and the clot disrupting section is in the clot. Then, the control unit is manipulated to control the clot disrupting section, to disrupt the clot. Finally, the control unit is used to dynamically position the deployed expandable device as it is removed from the artery, with clot fragments in the clot fragment guard section, whereby the clot fragments are removed from the patient.

In a third separate aspect, the present invention is a clot treatment device that includes a nitinol frame, having a compressed state, wherein the treatment device can fit within a cylinder of less than 1.5 mm internal diameter and 15 mm length and an expanded state, and that has a proximal portion and a distal portion along a longitudinal dimension. Also, the distal portion supports a reinforced, perforated silicone barrier, having a dimension transverse to the longitudinal dimension.

In addition to the exemplary aspects and embodiments described above, further aspects and embodiments will become apparent by reference to the drawings and by study of the following detailed descriptions.

### BRIEF DESCRIPTION OF THE DRAWINGS

Exemplary embodiments are illustrated in referenced drawings. It is intended that the embodiments and figures disclosed herein are to be considered illustrative rather than restrictive.

FIG. 1A is a perspective view of the angular artery, blocked by a clot, with the catheter assembly of the present invention threaded through the arterial system to the clot and the treatment device of the present invention deployed at the clot.

FIG. 1B is a perspective view of the angular artery, blocked by a clot, with the catheter assembly of the present invention threaded through the arterial system to the clot and the treatment device of the present invention at a further stage of treating said clot.

FIG. 1C is a perspective view of the angular artery, blocked by a clot, with the catheter assembly of the present invention threaded through the arterial system to the clot and the treatment device of the present invention removing clot fragments.

FIG. 2 shows the clot treatment device of FIG. 1A, in its undeployed state inside the tube of the catheter assembly of the present invention.

FIG. 3 shows the tube of the catheter assembly of the present invention, with the clot treatment device deployed out of it.

FIG. 4A shows a cross sectional view of the catheter tube of FIG. 3, in cross-section and the view-line 4A-4A.

FIG. 4B shows a cross sectional view of the catheter tube of FIG. 3, in cross-section and the view-line 4B-4B.

FIG. 4C shows a cross sectional view of the catheter tube of FIG. 3, in cross-section and the view-line 4C-4C.

FIG. 5 shows a perspective view of the full assembly, including the control assembly and showing the clot disruption device in deployed state.

FIG. 6 shows the control assembly of the catheter assembly of the present invention, in a neutral state.

FIG. 7 shows the distal end of the catheter assembly of the present invention, with the clot treatment device deployed in a neutral position.

FIG. 8 shows the control assembly of the catheter assembly of the present invention, in a state adapted to bend the clot treatment device in a first direction.

FIG. 9 shows the distal end of the catheter assembly of the present invention, with the clot treatment device deployed in a first direction.

FIG. 10 shows the control assembly of the catheter assembly of the present invention, in a state adapted to bend the clot treatment device in a second direction.

FIG. 11 shows the distal end of the catheter assembly of the present invention, with the clot treatment device deployed in a second direction.

FIG. 12 shows an isometric view of the clot treatment device of FIG. 1, in an unfinished state, during construction.

FIG. 13 shows an isometric view of the clot treatment device of FIG. 12, in a further state of being constructed.

FIG. 14 shows an isometric view of the clot treatment device of FIG. 13, in a finished state.

### DETAILED DESCRIPTION OF THE PREFERRED EMBODIMENTS

Definition: Upstream, as it is used in this application means further displaced in the direction of blood flow.

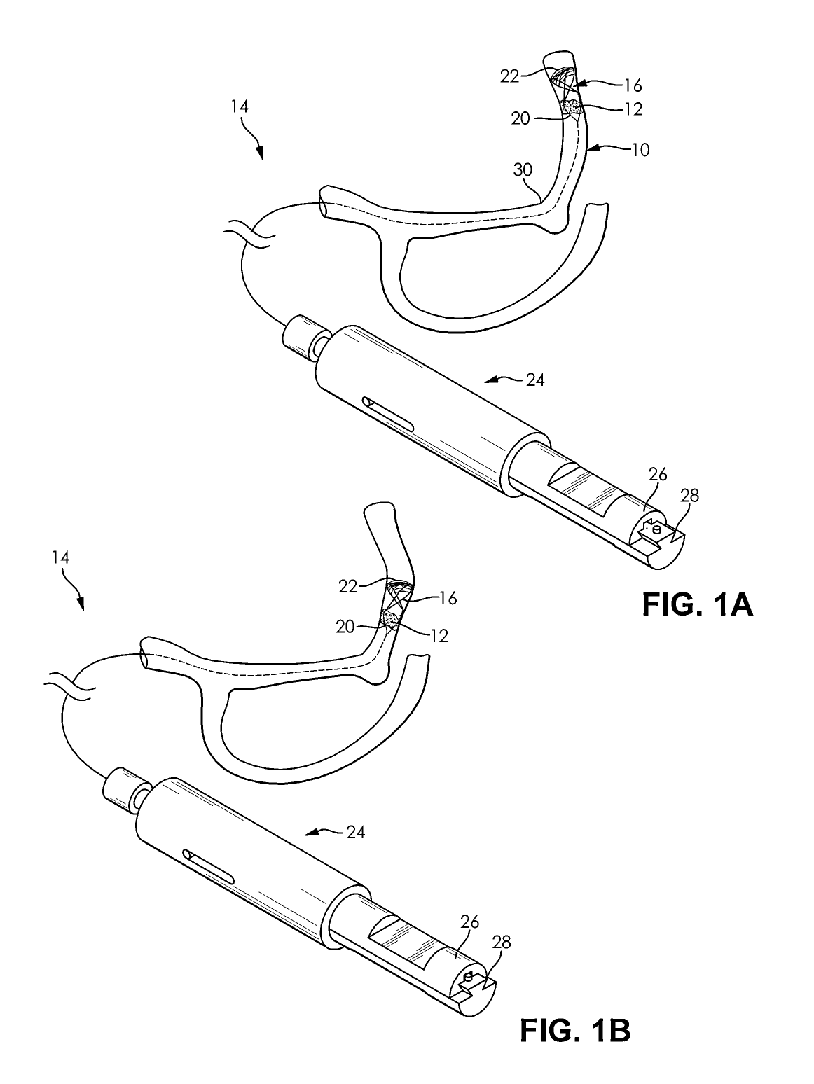

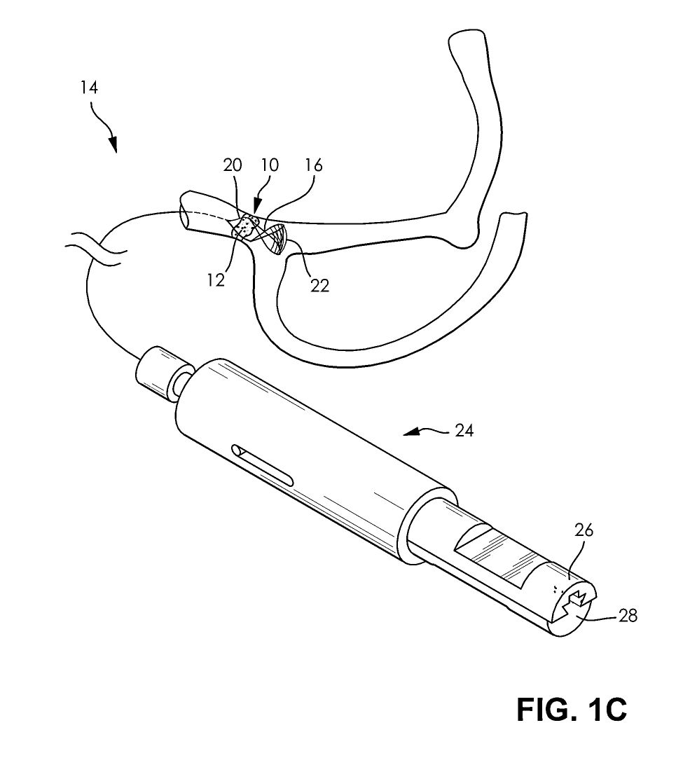

Referring to FIGS. 1A-1C, in a first preferred method, a blood vessel 10 (most typically an artery), that has been blocked by a clot 12, is addressed by a catheter assembly 14 that houses a clot disruption and removal device 16, which can be deployed as shown. The distal end of assembly 14 is introduced into the clot 12, and the device 16 is deployed into the clot 12, thereby penetrating through the clot and expanding out of the distal or upstream side of the clot. A proximal portion 20 of device 16 acts to disrupt the clot, whereas an upstream or distal portion 22 of device 16 opens into an umbrella structure to catch any clot fragments created by the clot disruption. Assembly 14 includes a control unit 24 that may be used to control device 16, and as will be explained further below, can be used to rotate device 16, thereby further disrupting clot 12. Control unit 24 can also be used to change the pitch of device 16, by the relative advancement of a first wire-handle 26 and a second wire-handle 28. Referring to FIGS. 1B and 1C, this quality is used after fragments have lodged in distal umbrella 22, to change the orientation of device 16, including umbrella 22, in order to be guided around a bend 30, out of vessel 10 in expanded form, while retaining the clot fragments.

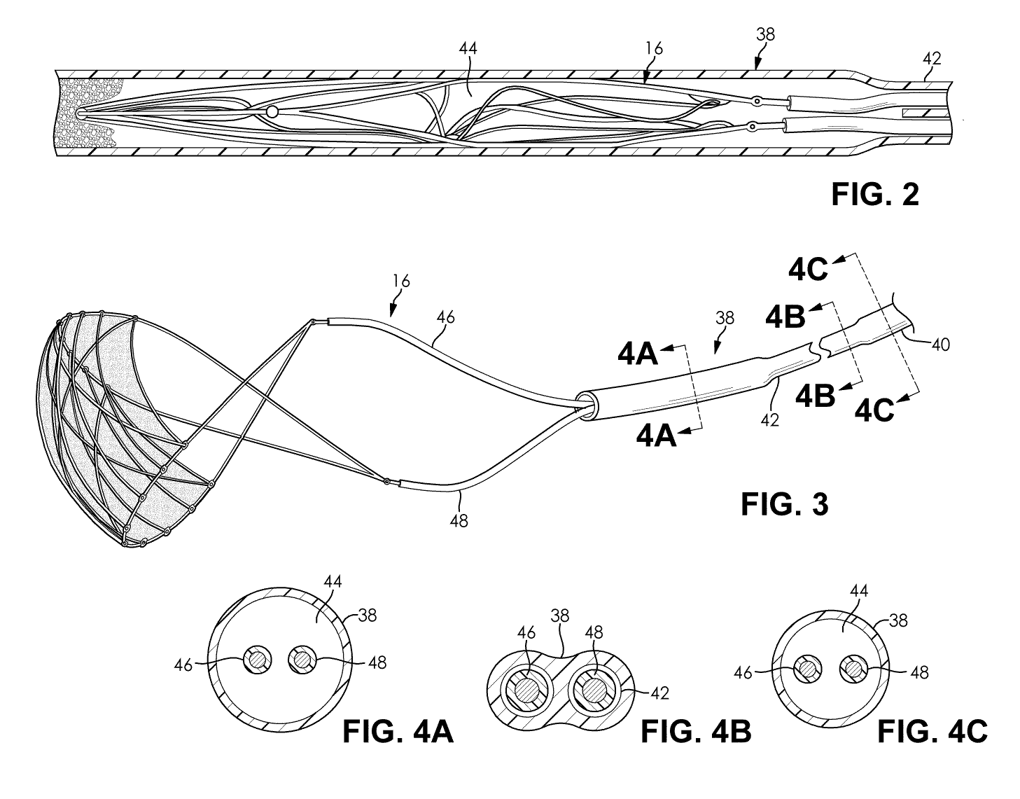

Referring to FIGS. 2-4C, assembly 14, distal to control unit 24, includes a flexible tube 38, which defines a proximal lumen 40, a medial split lumen 42, and a distal unitary and expanded lumen or fossa 44, which houses device 16 prior to deployment. A first wire 46 and a second wire 48, are attached to the first wire-handle 26 and a second wire-handle 28, respectively, and pass through lumens 40, 42 and 44. The tube 38 has an exterior diameter of about 1.5 mm, and a hydrophilic exterior surface, to aid in progressing toward a blood vessel destination.

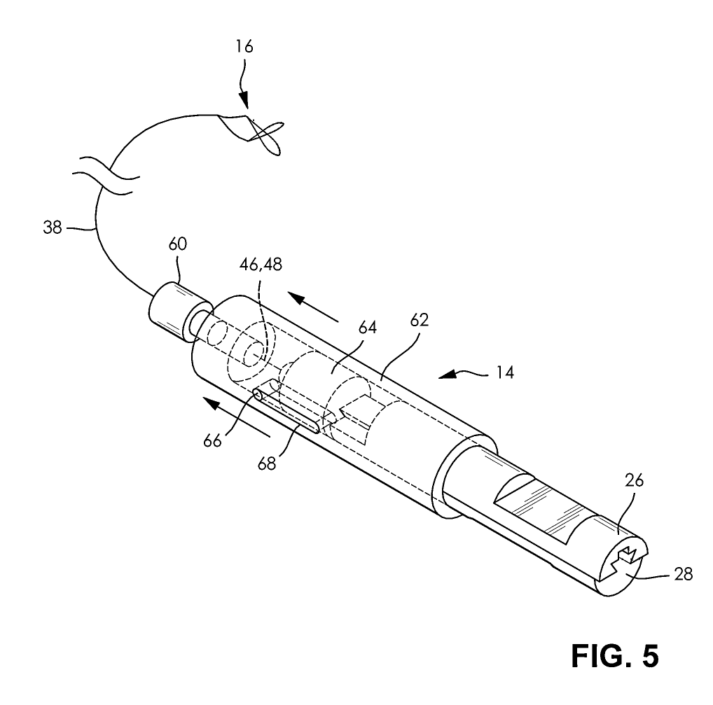

Referring now to FIG. 5, tube 38 is threaded through an end cap 60, and passes into a transparent chamber 62, where wires 46 and 48 emerge from tube 38, pass through a slider 64 and are separately anchored in handles 26 and 28, respectively. The travel extent of slider 64 is limited by a stop pin 66 and a slot 68.

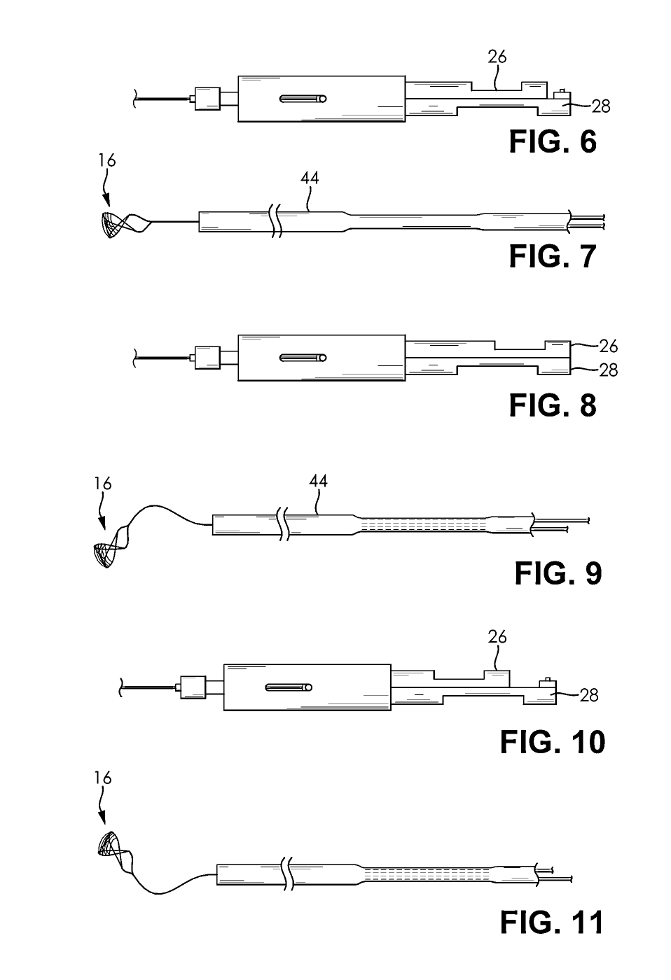

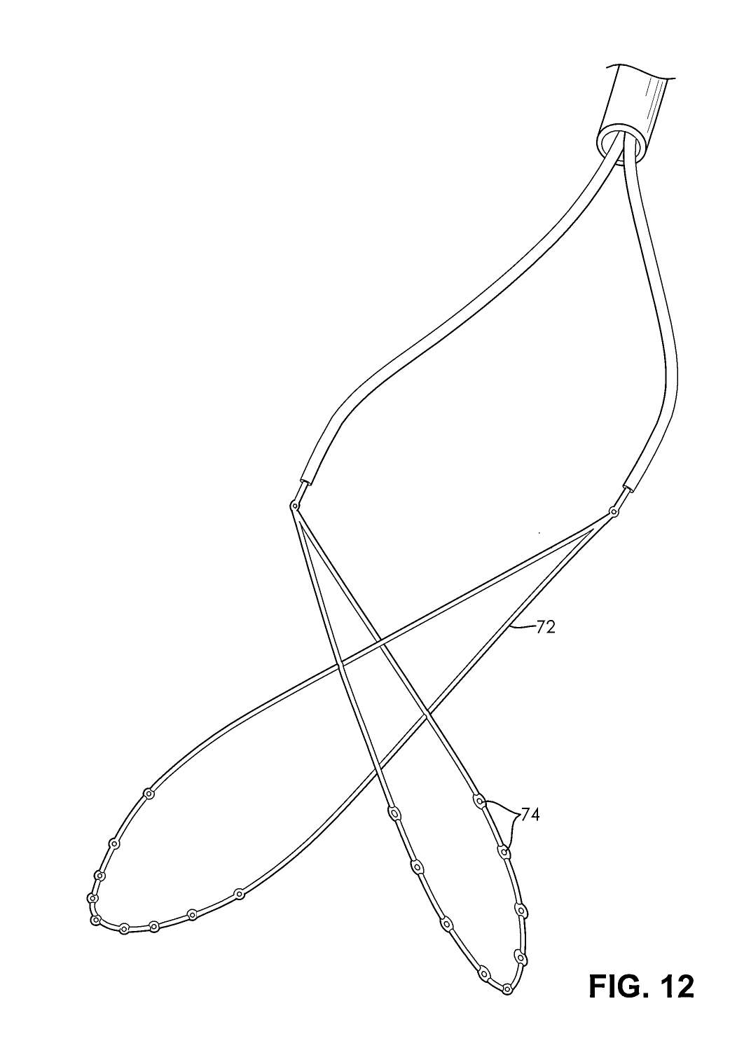

The double lumen section 42 shown in FIGS. 2 and 3 permits for the control of the shape and orientation of device 16 after it has been pushed out of fossa 44. As shown in FIGS. 6 through 11, after clot removal device 16 is pushed out of fossa 44, it bends toward wire handle 26, when handle 26 is retracted, and toward handle 28, when handle 28 is retracted. Device 16 can be rotated by rotating control unit 24. This freedom in positioning is most important during the retraction process, when as shown in FIGS. 1A through 1C, device 16 must be maneuvered around curves, such as bend 30, while in deployed, expanded form.

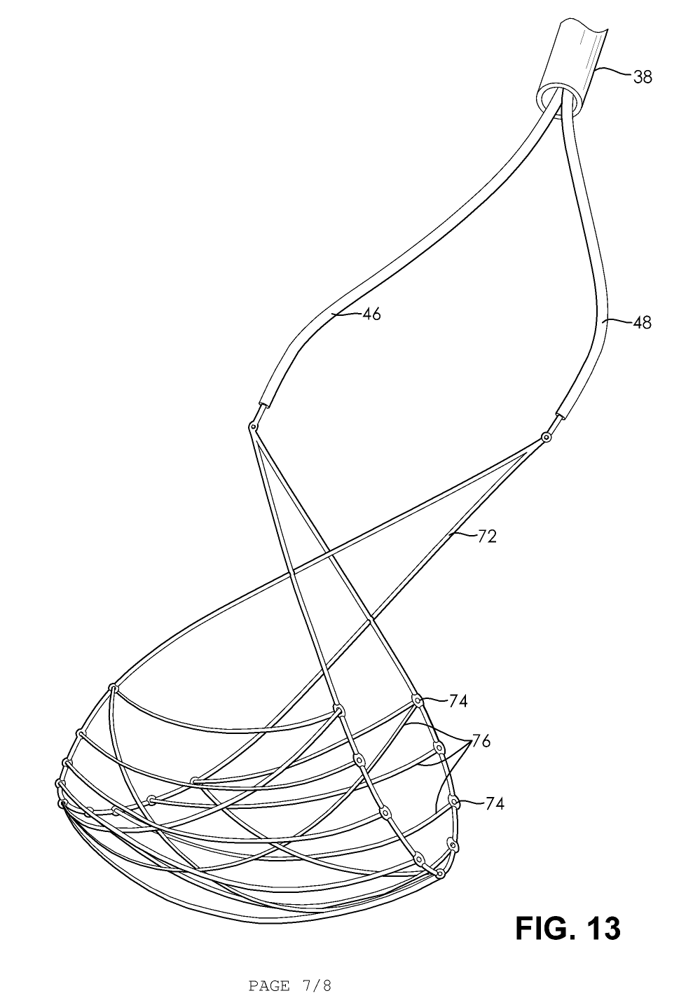

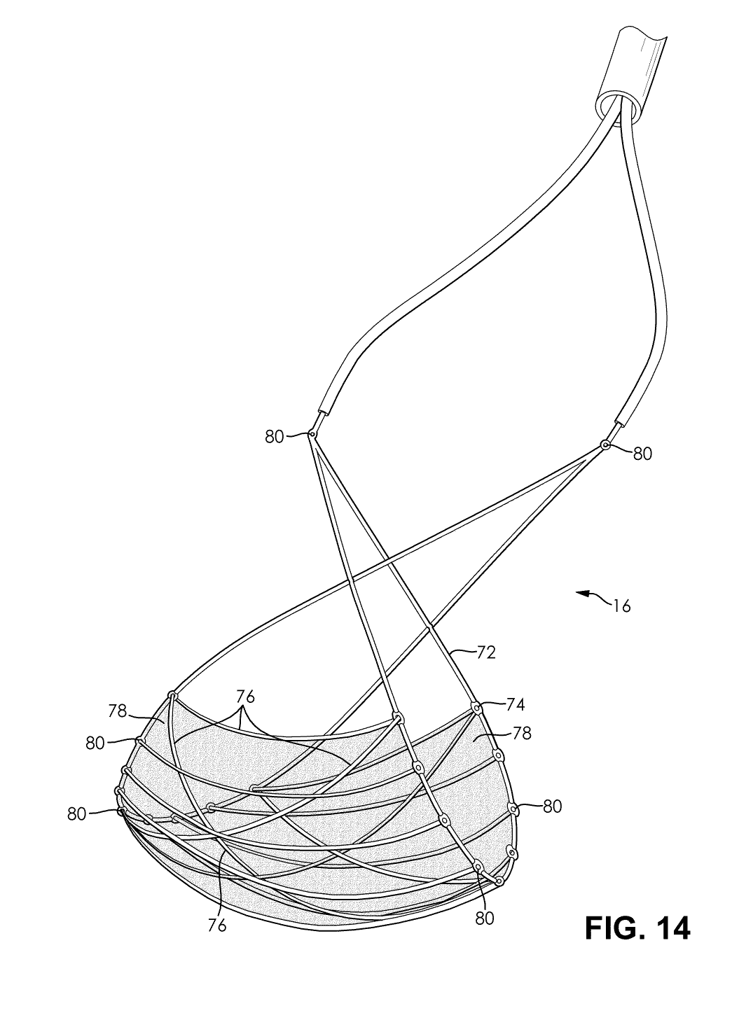

Referring to FIGS. 12 through 14, clot disruption and capture device 16 includes a wire frame 72, which is made of nitinol, or some other shape-memory material. Prior to use, device 16 is maintained at a temperature below human body temperature, thereby causing wire frame 72 to assume the shape shown in FIG. 14, when first pushed out of fossa 44. In another preferred embodiment, however, the natural spring force of the nitinol causes device 16 to expand when it is pushed out of fossa 44, and it retains this shape during positioning and use. Frame 72 defines a set of eyeholes 74, through which is threaded expanded poly tetrafluoroethylene (ePTFE) fibers 76, although in an alternative preferred embodiment a different thread material is used. A silicone barrier 78 is supported by frame 72 and fibers 76. In one preferred embodiment, silicone barrier is perforated to permit blood to flow through, while catching clot fragments. Silicone barrier 78 may be applied to frame 72 and fibers 76 and then cured in situ, or it may be cured in two sheets which are adhered (preferably with a silicone adhesive) together about fibers 76. Another feature of device 16 are a set of radio-opaque dots 80, placed to help a surgeon position device 16, during clot treatment.

Wires 46 and 48 are made of stainless steel alloy 304, which may also be referred to as alloy 18-8. This material is coated with poly tetrafluoroethylene. The nitinol alloy that frame 72 (FIG. 3) is made of is 54.5% to 57% nickel, with the remainder titanium, which forms a super-elastic alloy. The introducer tube 38 is made of high density polyethylene, coated at the distal tip with a hydrophilic coating. Finally, the silicone 78 of the device 16 is silicone MED 4820 or MED-6640, which is a high tear strength liquid silicone elastomer, having a Shore A durometer reading of 20-40. A MED6-161 Silicone Primer is used to attach silicone 78 to Nitinol frame 72.

While a number of exemplary aspects and embodiments have been discussed above, those possessed of skill in the art will recognize certain modifications, permutations, additions and sub-combinations thereof. It is therefore intended that the following appended claims and claims hereafter introduced are interpreted to include all such modifications, permutations, additions and sub-combinations as are within their true spirit and scope.

1. A medical catheter, comprising:
(a) a medial section having twin lumens, each enclosing a separately controllable wire;
(b) a control unit, proximal to said medial section, permitting a user to control said wires; and
(c) a fossa distal to said medial section, enclosing an expandable device that may be deployed to have a distal, clot fragment guard section and a proximal clot disrupting section and which is orientation controllable by said wires.

2. The medical catheter of claim 1, wherein said expandable device includes a wire frame.

3. The medical catheter of claim 2, wherein said clot fragment guard section includes a flexible barrier supported by said wire frame.

4. The medical catheter of claim 3, wherein said flexible barrier defines through-holes, thereby forming a filter for passing blood.

5. The medical catheter of claim 3, wherein said flexible barrier is made of silicone.

6. The medical catheter of claim 3, wherein said flexible barrier is reinforced by fibers.

7. The medical catheter of claim 6, wherein said fibers are made of expanded poly tetrafluoroethylene.

8. The medical catheter of claim 6, wherein said fibers are anchored to said wire frame.

9. The medical catheter of claim 6, wherein said wire frame includes eyeholes and said fibers are threaded through said eyeholes.

10. A medical procedure for treating a blood clot in an artery, comprising:
(a) providing a medical catheter, having:
(i) a distal end of said catheter, defining a fossa that encloses an expandable device that may be deployed to have a distal, clot fragment guard section and a proximal clot disrupting section; and
(ii) a control unit connected to said expandable device by a medial section, and permitting user deployment and position control of said expandable device, transmitted through said medial section;
(b) introducing said distal end of said catheter into said artery and guiding said distal end into said clot, deploying said expandable device from said fossa so that said clot fragment guard section is upstream from said clot and said clot disrupting section is in said clot;
(c) manipulating said control unit to control said clot disrupting section to disrupt said clot; and
(d) using said control unit to dynamically position said deployed expandable device as it is removed from said artery, with clot fragments in said clot fragment guard section, whereby said clot fragments are removed from said patient.

11. The method of claim 10, wherein said medial section includes a pair of parallel wires that are separately controllable by said control unit and wherein each one of said pair of wires is attached to a separate point on said expandable device, thereby permitting control of said wires by said control unit to result in position adjustment of said expandable device.

12. The method of claim 10, wherein said blood clot is formed in an artery in the brain, thereby constituting an ischemic stroke.

13. The method of claim 12, wherein said blood clot is formed in the angular artery.

14. The method of claim 10, wherein said expandable device is made of a wire frame, from which web material is suspended.

15. The method of claim 14, wherein said wire frame is made of nitinol wires.

16. The method of claim 10, wherein said step of guiding said distal end into said clot includes first introducing a guide catheter into said artery and threading said distal end through said guide catheter until it is in proximity to said clot.

17. The method of claim 16, wherein said step of introducing a guide catheter into said first artery, includes making an incision in the femoral artery, and extending said guide catheter through said femoral artery and any intermediate arteries to said first artery.

18. The method of claim 17, wherein said first artery is located in the brain.

19. The method of claim 18, wherein said first artery is the angular artery.

20. The method of claim 10, wherein said clot disrupting section is radially uneven in transverse dimension and is rotated to disrupt said clot.

21. The method of claim 20, wherein said control unit is connected to said expandable device by two wires attached at different points to said expandable device, said two wires extending through two separate, joined-together lumens in said medial section, thereby permitting a user to rotated said device by rotating said control unit, thereby disrupting said clot.

22. A clot treatment device, comprising:
(a) a nitinol frame, having a compressed state, wherein said treatment device can fit within a cylinder of less than 1.5 mm internal diameter and 15 mm length and an expanded state, having a proximal portion and a distal portion along a longitudinal dimension; and
(b) said distal portion supporting a reinforced, perforated silicone barrier, having a dimension transverse to said longitudinal dimension.

23. The clot treatment device of claim 22, wherein said silicone barrier is reinforced by expanded poly tetrafluoroethylene fiber.
